(19) United States
(12) Patent Application Publication
(10) Pub. No.: US 2015/0329484 A1
(43) Pub. Date: Nov. 19, 2015

(54) ESTER HAVING SULPHONE GROUP

(71) Applicants: Michael GRASS, Haltern am See (DE); Benjamin WOLDT, Bochum (DE); Andreas GEVERS, Bottrop (DE); Stefan BUCHHOLZ, Hanau (DE); Michael MEIER, Jockgrim (DE); Lucas MONTERO DE ESPINOSA, Karlsruhe (DE)

(72) Inventors: Michael GRASS, Haltern am See (DE); Benjamin WOLDT, Bochum (DE); Andreas GEVERS, Bottrop (DE); Stefan BUCHHOLZ, Hanau (DE); Michael MEIER, Jockgrim (DE); Lucas MONTERO DE ESPINOSA, Karlsruhe (DE)

(73) Assignee: Evonik Degussa GmbH, Essen (DE)

(21) Appl. No.: 14/654,295

(22) PCT Filed: Sep. 17, 2013
(86) PCT No.: PCT/EP13/69223

§ 371 Date: Jun. 19, 2015

(30) Foreign Application Priority Data
Dec. 19, 2012 (DE) 10 2012 223 673.2

(51) Int. Cl. C07C 317/44; C08K 5/41; C07C 315/02

(52) U.S. Cl. CPC C07C 317/44; C07C 315/02; C08K 5/41

(57) ABSTRACT

Esters having at least one sulphone group on the acid chain, processes for preparation thereof and use of the ester as a plasticizer for polymers.

The invention relates to an ester having at least one sulphone group on the acid chain.

The invention further relates to a process for preparing such an ester, and also to the use of the ester as a plasticizer for polymers.

U.S. Pat. No. 3,028,416 describes a sulphone ester in which the sulphone group has been incorporated into the acid chain.

The objective was firstly that of providing further esters which have one or more sulphone groups and have good plasticizer properties. A second objective was that of developing a preparation process for these esters.

The object is achieved by an ester according to Claim 1.

Ester of the formula I:

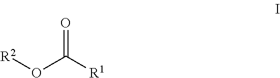

where R¹ is a carbon chain having 7 to 25 carbon atoms,
and the carbon chain R¹ has at least one sulphone group, and
R² is a linear or branched radical having 1 to 12 carbon atoms,
where the sulphur atom in the sulphone group is bonded directly to a carbon atom in the carbon chain R¹, and the carbon atom is not the carbon atom furthest removed from the C═O group, and where the sulphone group has the following structure:

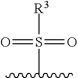

where R³ is an aliphatic radical having 1 to 8 carbon atoms.

Since the sulphone group cannot be bonded to the omega-carbon atom of the fatty acid or ester thereof, the sulphone group is thus not incorporated into the acid chain as in U.S. Pat. No. 3,028,416.

In one embodiment, the acid component of the ester was obtained from a vegetable oil. This can be illustrated, for example, by an ester mixture having an acid distribution as occurs in a vegetable oil. The source employed for the fatty acids may, for example, be soya oil or rapeseed oil or sunflower oil, but also other vegetable oils. The naturally occurring fatty acids/fatty acid mixtures are obtained, for example, by an ester cleavage or transesterification of the vegetable oils.

Preferably, the sulphone group(s) is/are localized on the carbon atoms having a double bond in naturally occurring unsaturated fatty acids, i.e. at positions 9, 10, 12, 13, 15 and 16 if the carbon chain R¹ has the corresponding length. The carbon C1 is the carbon of the C═O group.

In one embodiment of the invention, the R² is a linear or branched radical having 1 to 9 carbon atoms.

In one embodiment of the invention, R¹ is a carbon chain having 11 to 21 carbon atoms.

In one embodiment of the invention, R¹ is a carbon chain having 17 carbon atoms.

In one embodiment of the invention, R³ is an aliphatic radical having 1 to 4 carbon atoms.

In one embodiment of the invention, the carbon chain R¹ has exactly one sulphone group.

In one embodiment of the invention, the ester has the following structure:

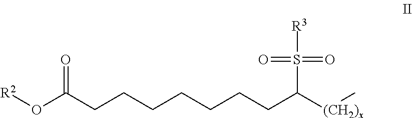

where x is a number from 6 to 12.

Preferably, x is a number from 8 to 10, more preferably 8.

In one embodiment of the invention, the ester has the following structure:

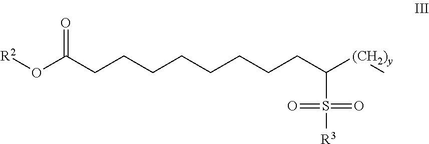

where y is a number from 5 to 11.

Preferably, y is a number from 7 to 9, more preferably 7.

In one embodiment, y has the value of x−1.

As well as the ester, an ester mixture is also claimed.

Ester mixture comprising a compound of the formula II and a compound of the formula III.

In one embodiment, the ratio of II to III is in the range from 55:45 to 45:55.

In one embodiment, the acid components of the ester mixture have been obtained from a vegetable oil. This can be illustrated, for example, by an ester mixture having an acid distribution as occurs in a vegetable oil. The source employed for the fatty acids may, for example, be soya oil or rapeseed oil, but also other vegetable oils. The naturally occurring fatty acids/fatty acid mixtures are obtained, for example, by an ester cleavage or transesterification of the vegetable oils.

As well as the ester and the ester mixture, the use thereof is also claimed.

Use of an above-described ester or ester mixture as a plasticizer for a polymer selected from: polyvinyl chloride, polyvinylidene chloride, polylactic acid, polyurethanes, polyvinyl butyral, polyalkyl methacrylates or copolymers thereof.

Use of an above-described ester or ester mixture as a plasticizer for polyvinyl chloride.

The inventive esters or ester mixtures can be used as a plasticizer for modification of polymers. These polymers are selected, for example, from the group consisting of:

Polyvinyl chloride (PVC), polyvinylidene chloride (PVDC), polyacrylates, especially polymethylmethacrylate (PMMA), polyalkylmethacrylate (PAMA), fluoropolymers, especially polyvinylidene fluoride (PVDF), polytetrafluoroethylene (PTFE), polyvinyl acetate (PVAc), polyvinyl alcohol (PVA), polyvinyl acetals, especially polyvinyl butyral (PVB), polystyrene polymers, especially polystyrene (PS), expandable polystyrene (EPS), acrylonitrile-styrene-acrylate (ASA), styrene-acrylonitrile (SAN), acrylonitrile-butadiene-styrene (ABS), styrene-maleic anhydride copolymer (SMA), styrene-methacrylic acid copolymer, polyolefins, especially polyethylene (PE) or polypropylene (PP), thermoplastic polyolefins (TPO), polyethylene-vinyl acetate (EVA), polycarbonates, polyethylene terephthalate (PET), polybutylene terephthalate (PBT), polyoxymethylene (POM), polyamide (PA), polyethylene glycol (PEG), polyurethane (PU), thermoplastic polyurethane (TPU), polysulphides (PSu), biopolymers, especially polylactic acid (PLA), polyhydroxybutyral (PHB), polyhydroxyvaleric acid (PHV), polyesters, starch, cellulose and cellulose derivatives, especially nitrocellulose (NC), ethyl cellulose (EC), cellulose acetate (CA), cellulose acetate/butyrate (CAB), rubber or silicones, and mixtures or copolymers of the polymers mentioned or monomeric units thereof. Preferably, the inventive polymers comprise PVC or homo- or copolymers based on ethylene, propylene, butadiene, vinyl acetate, glycidyl acrylate, glycidyl methacrylate, methacrylates, ethyl acrylates, butyl acrylates, or methacrylates having alkyl radicals of branched or unbranched alcohols having 1 to 10 carbon atom(s) bonded to the oxygen atom of the ester group, styrene, acrylonitrile or cyclic olefins.

Preferably, the polymer comprises, as a PVC type, suspension, bulk, microsuspension or emulsion PVC.

The esters and ester mixtures can also be used as fast gellers alongside other plasticizers.

Based on 100 parts by mass of polymer, the polymers contain preferably from 5 to 200, more preferably from 10 to 150, parts by mass of plasticizer.

The above-described esters or ester mixtures can be used in adhesives, sealing compounds, coating compounds, coating materials, paints, plastisols, foams, synthetic leathers, floor coverings (e.g. top layer), roof sheeting, underbody protection, fabric coatings, cables or wire insulations, hoses, extrusion articles, and in films, especially for automobile interiors, and also in carpets or inks.

The invention further provides mouldings or films comprising polymers comprising an inventive ester or ester mixture.

These mouldings or films are preferably a floor covering, a wall covering, a hose, a profile, a roofing sheet, a sealing sheet, a cable and wire sheath, a tarpaulin, an advertising banner, synthetic leather, packaging film, a medical article, a toy, a seal, a furnishing article. More preferably, the moulding or the film is a cable sheath for a high-temperature cable, or a constituent of automobile interior trim, especially a film for the dashboard.

The polymers comprising an inventive ester or ester mixture may, as well as the constituents mentioned, contain additives which are especially selected from the group consisting of: fillers, pigments, thermal stabilizers, UV stabilizers, antioxidants, viscosity regulators, flame retardants, lubricants, blowing agents, kickers.

The thermal stabilizers neutralize hydrochloric acid eliminated, inter alia, during and/or after the processing of the PVC and prevent thermal degradation of the polymer. Useful thermal stabilizers include all customary PVC stabilizers in solid and liquid form, for example based on Ca/Zn, Ba/Zn, Pb, Sn or organic compounds (OBS), and also acid-binding sheet silicates such as hydrotalcite. The mixtures may have a content of 0.5 to 12, preferably 1 to 10 and more preferably 1.5 to 8 parts by mass of thermal stabilizer per 100 parts by mass of polymer.

The pigments used in the context of the present invention may be either inorganic or organic pigments. The content of pigments is between 0.01 and 10% by mass, preferably 0.05 to 5% by mass, more preferably 0.1 to 3% by mass per 100 parts by mass of polymer. Examples of inorganic pigments are TiO₂, CdS, CoO/Al₂O₃, Cr₂O₃. Known organic pigments are, for example, azo dyes, phthalocyanine pigments, dioxazine pigments and aniline pigments.

The polymers comprising an inventive ester or ester mixture may comprise all fillers corresponding to the prior art. Examples of such fillers are mineral and/or synthetic and/or natural organic and/or inorganic materials, for example calcium oxide, magnesium oxide, calcium carbonate, barium sulphate, silicon dioxide, sheet silicate, industrial black, bitumen, wood (e.g. pulverized, in the form of pellets, microgranules, fibres etc.), paper, natural and/or synthetic fibres, etc. More preferably, at least one of the fillers used is a calcium carbonate or a calcium magnesium carbonate.

As well as the ester and the ester mixture, a process for preparation thereof is also claimed.

Process for preparing an above-described ester or ester mixture, comprising the process steps of:

a) initially charging an unsaturated fatty acid or fatty acid mixture or a fatty acid ester or fatty acid ester mixture,
b) adding a thiol onto the double bond, so as to form a thioether,
c) oxidizing the thioether to the sulphone,
d) esterifying the fatty acid or fatty acid mixture or transesterifying the fatty acid ester or fatty acid ester mixture.

In this process, process step d) can also be effected between process steps a) and b).

In one variant of the process, process step b) is effected with addition of a free-radical initiator. In this context, preference is given to azobis(isobutyronitrile) (AIBN).

In one variant of the process, process step c) is effected with addition of H₂O₂.

The invention is illustrated in detail hereinafter by working examples.

### 1. Synthesis Route

Proceeding from unsaturated fatty acids, the double bonds were S-functionalized, i.e. a sulphur-containing group was added onto the double bond, and it was possible to modify this further in subsequent steps.

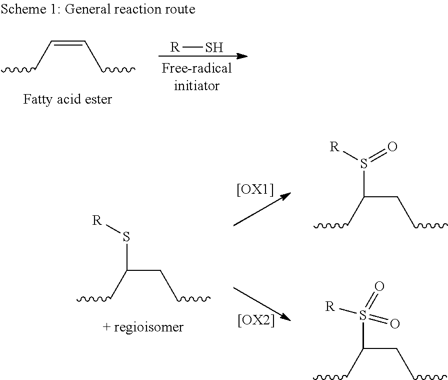

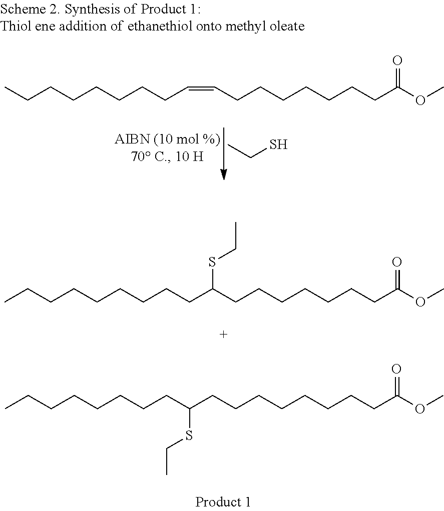

### 1.1 Thiol Ene Addition of Ethanethiol onto Methyl Oleate

First of all, methyl oleate was selected as the olefinic substrate, and the addition, initiated by azobis(isobutyronitrile) (AIBN), of ethanethiol was examined (Scheme 2). Methyl oleate was prepared by esterification of a commercially available oleic acid (90%, Aldrich, Mw=282.46 g/mol) having a purity of 90% with methanol. The AIBN-initiated free-radical reactions were conducted at 60 to 80° C. in order to obtain a good and constant concentration of free radicals within a time frame of 8 to 10 h. The thiol ene addition was conducted at 70° C., i.e. a temperature 35° C. above the boiling point of ethanethiol. Therefore, to avoid thiol losses in the course of the reaction, a high-pressure reactor (Berghof® BR-300) was used. Various equivalent ratios of thiol (5, 8 and 10 eq.) and of AIBN (0.02, 0.05 and 0.1) to double bond were tested: 10 eq. of thiol and 0.1 eq. of AIBN gave complete double bond conversion within 10 h of reaction at 700° C. (monitoring by means of GC-MS). The reaction mixture ultimately obtained contained Product 1 together with the thiol excess and diethyl disulphide (3% by weight of the overall thiol) as the sole by-product. The thiol excess and the by-product were easily removable by fractional distillation, which enabled recycling of the thiol. The purity of Product 1 can be regarded as the same as that of the methyl oleate starting material, since no further purification was required (i.e. 90%, since the methyl oleate used was 90% pure; the 10% impurities were saturated and polyunsaturated fatty acids). The structure of Product 1 was confirmed by GC-MS, ¹H NMR and ¹³C NMR, and the thermal properties thereof were analysed by means of DSC.

### 1.2 Oxidation of the Thioether Group

The oxidation of the thioether group in Product 1 was conducted with hydrogen peroxide (30% by volume aqueous solution). Even though Product 1 is insoluble in the hydrogen peroxide solution, the oxidation proceeds quantitatively when the suspension formed is stirred. It was found that hydrogen peroxide is capable of selectively oxidizing the thioether to a sulphoxide or a sulphone according to the reaction temperature. For instance, when the mixture was stirred at ° C. over a period of 20 h, the sulphoxide derivative (intermediate oxidation product) was obtained, and, when the mixture was heated to 70° C. over a period of 10 h, the sulphone 25 derivative (fully oxidized product) was obtained (Scheme 3). The reactions were monitored by means of ¹H NMR spectroscopy. On completion, the stirrer was switched off, whereupon two layers formed, which were easily separable. The product was washed with sodium sulphite to remove hydrogen peroxide traces remaining. No further purification was carried out.

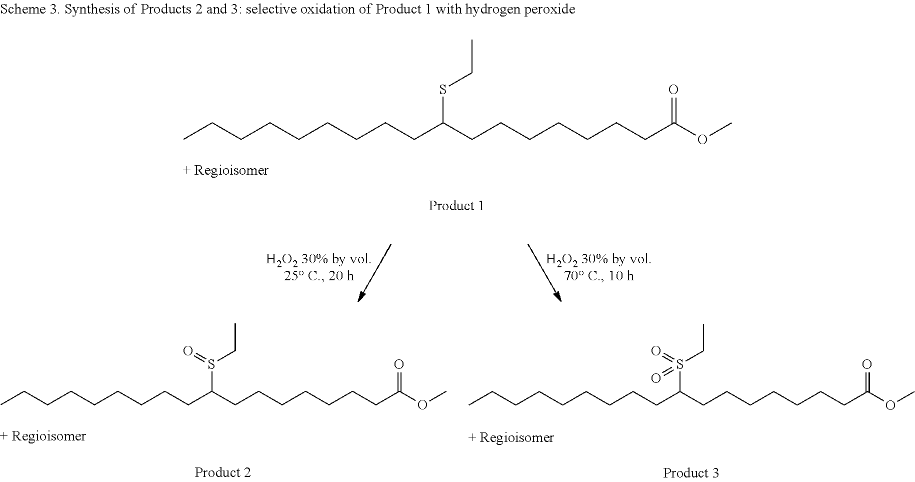

Alternatively, the urea-hydrogen peroxide complex (UHP) was tested as an oxidizing agent. UHP was examined since it is a very easy-to-handle white powder; however, the stirring of the reaction mixture necessitated heating thereof beyond the melting point of UHP (80° C.). As a result, the oxidation was not selective, and either a mixture of sulphoxide and sulphone derivative or only the sulphone derivative was obtained. Therefore, hydrogen peroxide was used in further oxidation reactions.

The DSC analysis of Product 2 (sulphoxide) showed a thermal decomposition which set in at 135° C. In the analysis of the remaining residue by means of ¹H NMR, a signal in the double bond range was observed. This can be explained by the thermolysis of the sulphoxide group to form an olefin and a sulphenic acid. The GC-MS analysis of Products 2 and 3 was impossible because of the low volatility thereof. The structures of Products 2 and 3 were confirmed by FAB-MS, ¹H NMR and ¹³C NMR, and the thermal properties thereof were analysed by means of DSC.

### 1.3 Increasing the Sulphur Content Per Molecule

The sulphur content per fatty acid ester was increased by using methyl linoleate as the olefinic substrate.

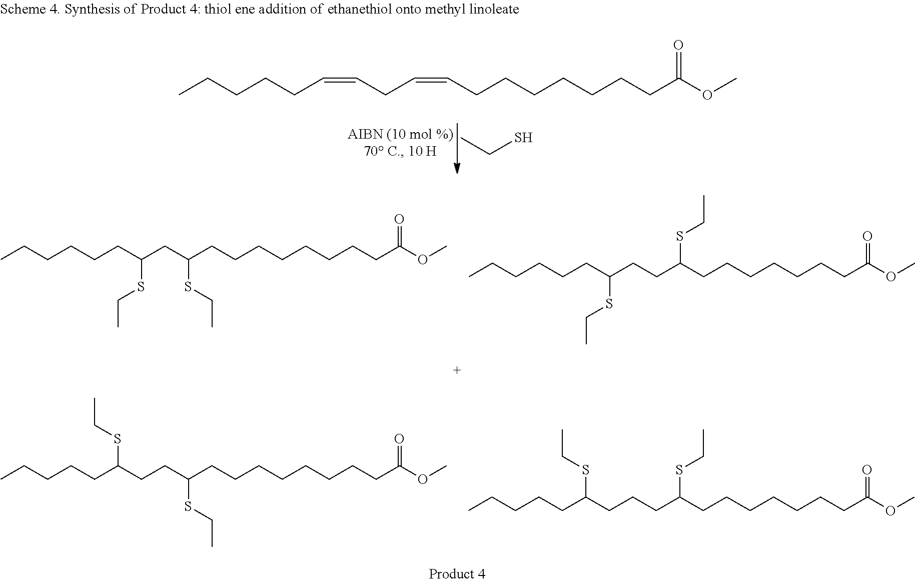

Methyl linoleate was prepared by esterifying a commercially available technical linoleic acid (60-74%, Aldrich, Mw=280.45 g/mol) with methanol. The thiol ene addition was conducted under the same reaction conditions as in the synthesis of Product 1, and the thiol excess and the disulphide by-product were likewise removed by fractional distillation. The purity of the resulting Product 4 can be regarded as the same as that of the methyl linoleate starting material, since no further purification was required. The structure of Product 4 was confirmed by GC-MS, ¹H NMR and ¹³C NMR, and the thermal properties thereof were analysed by means of DSC.

The oxidation of Product 4 to the sulphoxide derivative (Product 5) and sulphone derivative (Product 6) (Scheme 5) was conducted under the same reaction conditions as described for the synthesis of Products 2 and 3.

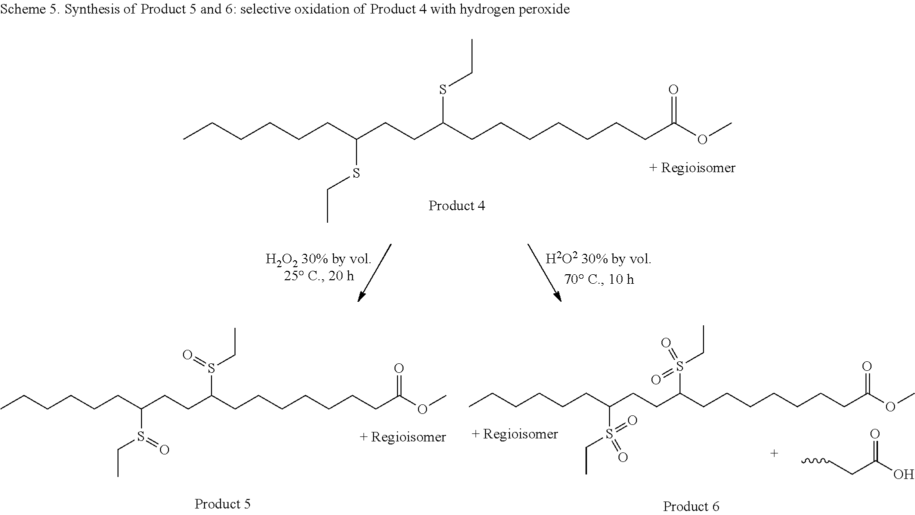

### 1.4 Extending the Thiol Alkyl Chain

The length of the alkyl chain of the thiol was varied from two carbon atoms (ethanethiol) to four and six carbon atoms by using butanethiol and hexanethiol.

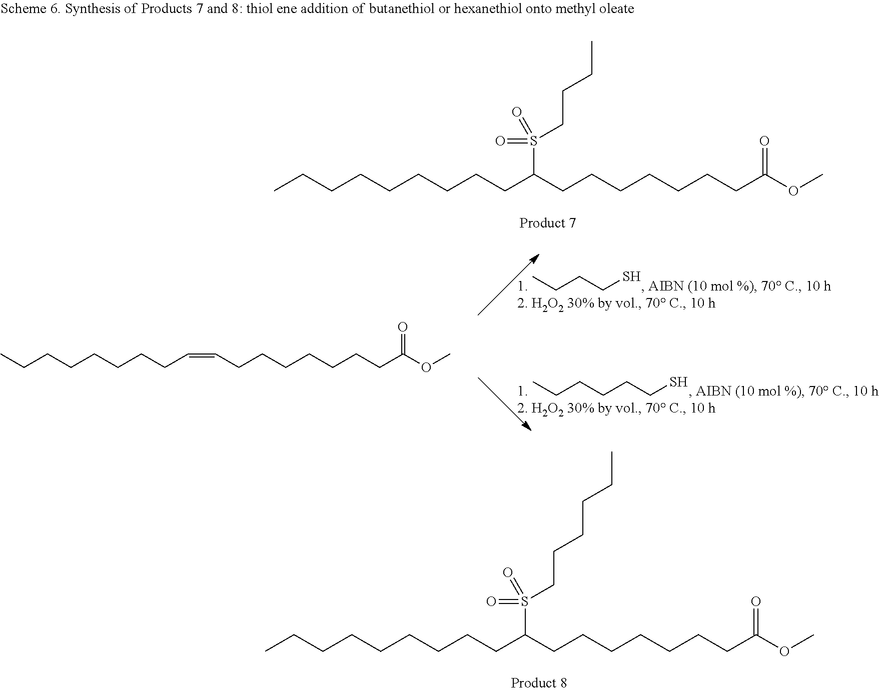

The olefinic substrate used was methyl oleate, and the reaction conditions were the same as for the synthesis of Product 1. The oxidation of the thioether function to the sulphone was likewise conducted in the same way as for the synthesis of Product 3. The GC-MS analysis of Products 7 and 8 was impossible because of their low volatility. The structures of Products 7 and 8 were confirmed by FAB-MS, ¹H NMR and ¹³C NMR, and the thermal properties thereof were analysed by means of DSC.

### 2. Experiments

### 2.1 Materials

Oleic acid (90%, Aldrich, Mw=282.46 g/mol), linoleic acid (60-74%, Aldrich, Mw=280.45 g/mol), trimethyl orthoformate (99%, Aldrich, Mw=106.12 g/mol, density=0.970 g/ml), sulphuric acid (95-98%, Mw=98.08 g/mol, density=1.840 g/ml), ethanethiol (97%, Aldrich, Mw=62.13 g/mol, density=0.839 g/ml), butanethiol (99%, Aldrich, Mw=90.19 g/mol, density=0.842 g/ml), hexanethiol (95%, Aldrich, Mw=118.24 g/mol, density=0.832 g/ml, 500 ml), hydrogen peroxide, 30% by volume, Na₂SO₄ (Aldrich, anhydrous), 2,2′-azobis(2-methylpropionitrile) (AIBN, 98%, Aldrich, Mw=164.21 g/mol). All the solvents used were of technical grade quality.

### 2.2 Methods

¹H NMR spectra were recorded in CDC₃, DMSO-d6 or CD₃OD on a Bruker AVANCE DPX spectrometer at 300 MHz. Chemical shifts (δ) are reported in parts per million relative to the tetramethylsilane internal standard (TMS, δ=0.00 ppm).

GC-MS (El) chromatograms were recorded on a Varian 431 GC instrument with a FactorFour™ VF-5 ms capillary column (30 m×0.25 mm×0.25 μm) and a Varian 210 ion trap mass detector. Scans were conducted from 40 to 650 mlz at a rate of 1.0 Scan×s⁻¹. The following oven temperature program was used: start temperature 95° C., hold for 1 min, heat at ° C.×min⁻¹ to 200° C., hold for 2 min, heat at 15° C.×min⁻¹ to 325° C., hold for 5 min. The temperature of the injector transfer line was set to 250° C. The measurements were conducted in split-split mode (split ratio 50:1) with helium as carrier gas (flow rate 1.0 ml×min⁻¹).

DSC experiments (DSC=Differential Scanning Calorimetry) were conducted on a DSC821e calorimeter (Mettler Toledo) under a nitrogen atmosphere at a heating rate of 10° C.×min⁻¹ up to a temperature of 150° C. and with a sample mass of about 5 mg.

FAB mass spectra (FAB=Fast Atom Bombardment) were measured with a Finnigan MAT95 instrument.

### 2.3 Synthesis and Purification Methods

### 2.3.1 Synthesis of Methyl Oleate

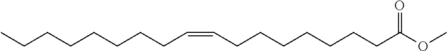 [SMILES: CCCCCCCC/C=C\CCCCCCCC(=O)OC]

Chemical formula: C₁₉H₃₆O₂
Molecular weight: 296.49 g/mol
Oleic acid (350 g, 1.24 mol), methanol (350 ml, 8.65 mol), trimethyl orthoformate (50 ml, 0.45 mol) and sulphuric acid (10 ml, 0.19 mol) were mixed in a 1 l round-bottom flask and heated to reflux while stirring for 10 h. The reaction mixture was washed with diethyl ether and water (in the case of poor phase separation with addition of sodium chloride solution), 10% by weight aqueous Na₂CO₃ solution and water. The organic fraction was dried over anhydrous Na₂SO₄ and filtered, and then the solvent was drawn off on a rotary evaporator under reduced pressure. For removal of coloured impurities, the product was filtered through silicon dioxide with hexane, after which methyl oleate was obtained in the form of a very pale yellowish liquid in quantitative yield. The analytical data correspond to those of the commercially available product.

Composition according to GC-MS: methyl linoleate (59.47%), methyl oleate (33.47%), methyl palmitate (5.26%), methyl stearate (1.80%).

### 2.3.2 Synthesis of Methyl Linoleate

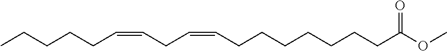 [SMILES: CCCCC/C=C\C/C=C\CCCCCCCC(=O)OC]

Chemical formula: C₁₉H₃₄O₂
Molecular weight: 294.47 g/mol
Linoleic acid (350 g, 1.25 mol), methanol (350 ml, 8.65 mol), trimethyl orthoformate (50 ml, 0.45 mol) and sulphuric acid (10 ml, 0.19 mol) were mixed in a 1 l round-bottom flask and heated to reflux while stirring for 10 h. The reaction mixture was washed with diethyl ether and water (in the case of poor phase separation with addition of sodium chloride solution), 10% by weight aqueous Na₂CO₃ solution and water. The organic fraction was dried over anhydrous Na₂SO₄ and filtered, and then the solvent was drawn off on a rotary evaporator under reduced pressure. For removal of coloured impurities, the product was filtered through silicon dioxide with hexane, after which methyl linoleate was obtained in the form of a pale yellowish liquid in quantitative yield. The analytical data correspond to those of the commercially available product.

Composition according to GC-MS: methyl oleate (96.23%), methyl palmitate (1.92%), methyl stearate (1.85%).

### 2.3.3 Synthesis of a mixture of methyl 9-(ethylthio)octadecanoate/methyl 10-(ethylthio)octadecanoate (product 1)

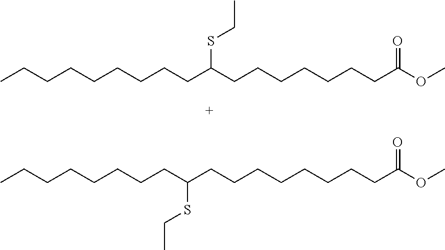

Chemical formula: C₂₁H₄₂O₂S
Molecular weight: 358.62 g/mol
Methyl oleate (80 g, 0.27 mol), ethanethiol (200 ml, 2.7 mol) and AIBN (4.4 g, 0.027 mol) were introduced into a Berghof® BR-300 600 ml pressure reactor and heated to 70° C. for 10 h. The reactor was allowed to cool to room temperature and then opened. The reaction mixture, a yellowish liquid, was transferred into a round-bottom flask and connected to a distillation apparatus. The ethanethiol excess and the by-product (diethyl disulphide) were distilled off under standard pressure and collected in separate fractions. The remaining traces of diethyl disulphide were removed by distillation under reduced pressure (20 mbar) and 150° C. over a period of 1 h. The remaining orange product was filtered through a 2 cm layer of silicon dioxide using hexane as an eluent. After drawing off the hexane on a rotary evaporator under reduced pressure, Product 1 was obtained in the form of an orange liquid in quantitative yield.

Composition according to GC-MS: Product 1 (97.7%), methyl palmitate (0.97%), methyl stearate (0.91%), unidentified unsaturated fatty acid ester (0.46%).

Melting points: −26° C. and −12° C.

¹H NMR (300 MHz, CDCl₃, δ in ppm): δ=3.64 (s, OMe), 2.61-2.46 (m, —S—CH<), 2.48 (q, J=7.70 Hz, CH₃—CH₂—S—), 2.28 (t, J=7.50 Hz, —CH₂—CO—), 1.66-1.55 (m, —CH₂—CH₂—CO—), 1.55-1.44 (m, —CH₂—CH(S)—CH₂—), 1.44-1.13 (m, —CH₂— and CH₃—CH₂—S—), 0.86 (t, J=6.70 Hz, —CH₃).

### 2.3.4 Synthesis of a mixture of methyl 9-(ethylsulphinyl)octadecanoate/methyl 10-(ethylsulphinyl)octadecanoate (Product 2)

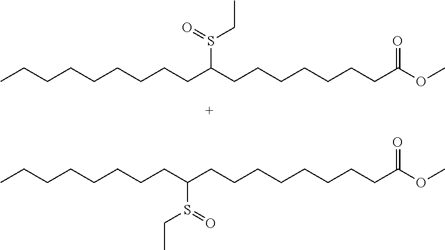

Chemical formula: C₂₁H₄₂O₃S

Molecular weight: 374.62 g/mol

Product 1 (150 g, 0.4 mol) and 30% by volume hydrogen peroxide (91 ml, 0.8 mol) were mixed in a 500 ml round-bottom flask and stirred vigorously at room temperature for 20 h. The reaction mixture was transferred into a separating funnel and admixed with ethyl acetate (150 ml) and sodium chloride solution (150 ml). The aqueous layer was extracted and the organic layer was washed with 10% by weight aqueous Na₂SO₃ solution until the hydrogen peroxide content was below the detection limit of the Quantofix® peroxide test (below 1 mg/l). If necessary, for better phase separation, additional sodium chloride solution was added. The organic layer was dried over anhydrous Na₂SO₄ and filtered (if the solution remains turbid, it can be filtered through a 2 cm layer of silicon dioxide, in which case it is washed through with ethyl acetate), and then the solvent was drawn off on a rotary evaporator under reduced pressure. Product 2 was obtained in the form of a yellowish liquid.

FAB-MS [M+H]⁺ calculated: 375.62. found: 375.4.

Melting point: −4° C.

Thermal decomposition: endothermic transition commencing at about 135° C. with a maximum at 186° C.

¹H NMR (300 MHz, CDCl₃, δ in ppm): δ=3.63 (s, OMe), 2.59 (q, J=7.40 Hz, CH₃—CH₂—S(O)—), 2.49-2.39 (m, —S(O)—CH<), 2.27 (t, J=7.40 Hz, —CH₂—CO—), 1.77-1.48 (m, —CH₂—CH₂—CO— and —CH₂—CHS(O)—CH₂—), 1.48-1.14 (m, —CH₂— and CH₃—CH₂—S(O)—), 0.84 (t, J=6.00 Hz, —CH₃).

### 2.3.5 Synthesis of a mixture of methyl 9-(ethylsulphonyl)octadecanoate/methyl 10-(ethylsulphonyl)octadecanoate (Product 3)

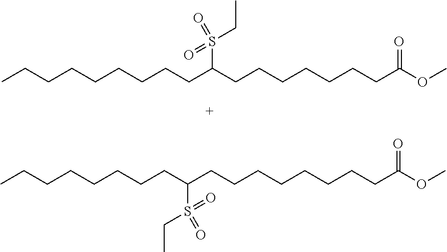

Chemical formula: C₂₁H₄₂O₄S

Molecular weight: 390.62 g/mol

Product 1 (150 g, 0.4 mol) and 30% by volume hydrogen peroxide (182 ml, 1.6 mol) were mixed in a 500 ml round-bottom flask and stirred vigorously at 70° C. for 10 h. The reaction mixture was transferred into a separating funnel and admixed with ethyl acetate (150 ml) and sodium chloride solution (150 ml). The aqueous layer was extracted and the organic layer was washed with 10% by weight aqueous Na₂SO₃ solution until the hydrogen peroxide content was below the detection limit of the Quantofix® peroxide test (below 1 mg/l). If necessary, for better phase separation, additional sodium chloride solution was added. The organic layer was dried over anhydrous Na₂SO₄ and filtered (if the solution remains turbid, it can be filtered through a 2 cm layer of silicon dioxide, in which case it is washed through with ethyl acetate), and then the solvent was drawn off on a rotary evaporator under reduced pressure. Product 3 was obtained in the form of a very pale yellowish liquid.

FAB-MS [M+H]⁺ calculated: 391.62. found: 391.3.

Melting point: 0° C.

¹H NMR (300 MHz, CDCl₃, δ in ppm): δ=3.64 (s, OMe), 2.93 (q, J=7.40 Hz, CH₃—CH₂—S(O)₂—), 2.81-2.72 (m, —S(O)₂—CH<), 2.28 (t, J=7.40 Hz, —CH₂—CO—), 1.94-1.80 (m, —CH^{A}H^{B}—CHS(O)₂—CH^{A}H^{B}—), 1.70-1.53 (m, —CH^{A}H^{B}—CHS(O)₂—CH^{A}H^{B}— and —CH₂—CH₂—CO—), 1.36 (t, J=7.50 Hz, CH₃—CH₂—S(O)₂—), 1.53-1.17 (m, —CH₂—), 0.86 (t, J=6.00 Hz, —CH₃).

### 2.3.6 Synthesis of a mixture of methyl 9,12-bis(ethylthio)octadecanoate/methyl 9,13-bis(ethylthio)octadecanoate/methyl 10,12-bis(ethylthio)octadecanoate/methyl 10,13-bis(ethylthio)octadecanoate (Product 4)

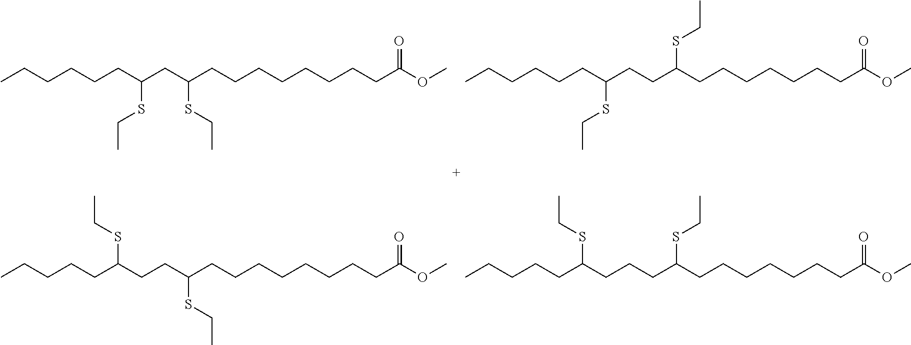

Chemical formula: C₂₃H₄₆O₂S₂

Molecular weight: 418.74 g/mol

Methyl linoleate (80 g, 0.27 mol), ethanethiol (300 ml, 4.05 mol) and AIBN (6.6 g, 0.041 mol) were introduced into a Berghof® BR-300 600 ml pressure reactor and heated to 70° C. for 10 h. The reactor was allowed to cool to room temperature and then opened. The reaction mixture, a yellowish liquid, was transferred into a round-bottom flask and connected to a distillation apparatus. The ethanethiol excess (36° C.) and the by-product (diethyl disulphide, 150° C.) were distilled off under standard pressure and collected as separate fractions. The remaining traces of diethyl disulphide were removed by distillation under reduced pressure (20 mbar) at 150° C. over a period of 1 h. The remaining orange product was filtered through a 2 cm layer of silicon dioxide and washed through with hexane. After the hexane had been drawn off on a rotary evaporator under reduced pressure, Product 4 was obtained in the form of a pale orange liquid in quantitative yield.

Composition according to GC-MS (see FIG. 16): Product 4 (66.6%), Product 1 (29.4%), methyl palmitate (2.8%), methyl stearate (1.2%).

Melting point: −10° C.

¹H NMR (300 MHz, CDCl₃, δ in ppm): δ=3.65 (s, OMe), 2.61-2.44 (m, CH₃—CH₂—S—CH<), 2.28 (t, J=7.50 Hz, —CH₂—CO—), 1.69-1.45 (m, —CH₂—CH₂—CO— and —CH₂—CH(S)—CH₂—), 1.45-1.16 (m, —CH₂— and CH₃—CH₂—S—), 0.91-0.82 (m, —CH₃).

### 2.3.7 Synthesis of a mixture of methyl 9,12-bis(ethylsulphinyl)octadecanoate/methyl 9,13-bis(ethylsulphinyl)octadecanoate/methyl 10,12-bis(ethylsulphinyl)octadecanoate/methyl 10,13-bis(ethylsulphinyl)octadecanoate (Product 5)

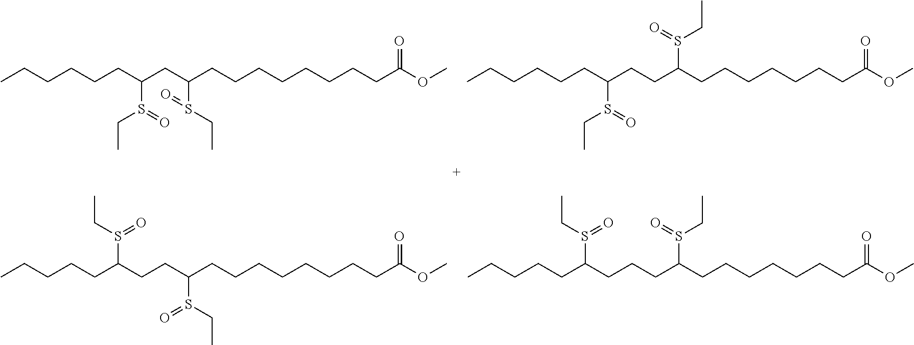

Chemical formula: C₂₃H₄₆O₄S₂

Molecular weight: 450.74 g/mol

Product 4 (150 g, 0.36 mol) and 30% by volume hydrogen peroxide (125 ml, 1.08 mol) were mixed in a 500 ml round-bottom flask and stirred vigorously at room temperature for 20 h. The reaction mixture was transferred into a separating funnel and admixed with ethyl acetate (200 ml) and sodium chloride solution (200 ml). The aqueous layer was extracted and the organic layer was washed with 10% by weight aqueous Na₂SO₃ solution until the hydrogen peroxide content was below the detection limit of the Quantofix® peroxide test (below 1 mg/l). If necessary, for better phase separation, additional sodium chloride solution was added. The organic layer was dried over anhydrous Na₂SO₄ and filtered (if the solution remains turbid, it can be filtered through a 2 cm layer of silicon dioxide, in which case it is washed through with ethyl acetate), and then the solvent was drawn off on a rotary evaporator under reduced pressure. Product 5 was obtained in the form of a yellowish liquid.

FAB-MS [M+H]⁺ calculated: 451.74. found: 451.2. Also found: 375.2 (Product 2 as a result of the presence of methyl oleate in the technical methyl linoleate which serves as the starting material).

Melting point: 5° C.

Thermal decomposition: endothermic transition commencing at about 162° C. with a maximum at 190° C.

¹H NMR (300 MHz, CDCl₃, δ in ppm): δ=3.65 (s, OMe), 2.61 (q, J=7.40 Hz, CH₃—CH₂—SO—), 2.54-2.42 (m, —SO—CH<), 2.28 (t, J=7.50 Hz, —CH₂—CO—), 1.76-1.51 (m, —CH₂—CH₂—CO— and —CH₂—CHS(O)—CH₂—), 1.51-1.18 (m, —CH₂— and CH₃—CH₂—S—), 0.90-0.81 (m, —CH₃).

### 2.3.8 Synthesis of a mixture of methyl 9,12-bis(ethylsulphonyl)octadecanoate/methyl 9,13-bis(ethylsulphonyl)octadecanoate/methyl 10,12-bis(ethylsulphonyl)octadecanoate/methyl 10,13-bis(ethylsulphonyl)octadecanoate (Product 6)

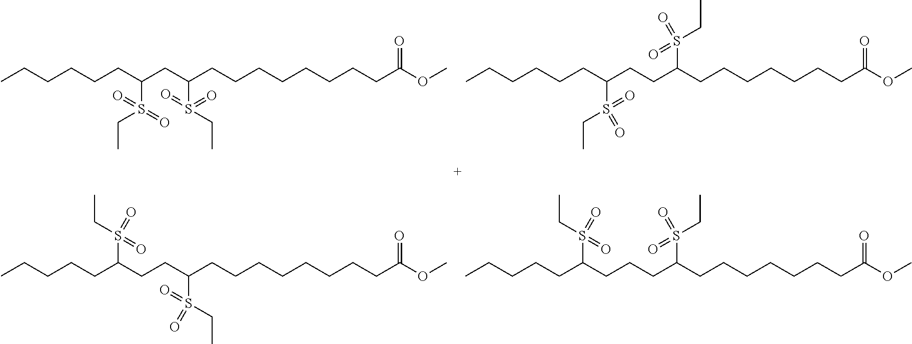

Chemical formula: C₂₃H₄₆O₆S₂

Molecular weight: 482.74 g/mol

Product 4 (150 g, 0.36 mol) and 30% by volume hydrogen peroxide (245 ml, 2.16 mol) were mixed in a 1 l round-bottom flask and stirred vigorously at 70° C. for 10 h. The reaction mixture was transferred into a separating funnel and admixed with ethyl acetate (200 ml) and sodium chloride solution (200 ml). The aqueous layer was extracted and the organic layer was washed with 10% by weight aqueous Na₂SO₃ solution until the hydrogen peroxide content was below the detection limit of the Quantofix® peroxide test (below 1 mg/l). If necessary, for better phase separation, additional sodium chloride solution was added. The organic layer was dried over anhydrous Na₂SO₄ and filtered (if the solution remains turbid, it can be filtered through a 2 cm layer of silicon dioxide, in which case it is washed through with ethyl acetate), and then the solvent was drawn off on a rotary evaporator under reduced pressure. Product 6 was obtained in the form of a pale yellowish liquid.

FAB-MS [M+H]⁺ calculated: 483.74. found: 483.2. Also 469.2 (form of the free acid as a result of hydrolysis during the oxidation).

Melting points: broad range of melting transitions between −69° C. and 11° C.

### 2.3.9 Synthesis of a mixture of methyl 9-(butylsulphonyl)octadecanoate/methyl 10-(butylsulphonyl)octadecanoate (Product 7)

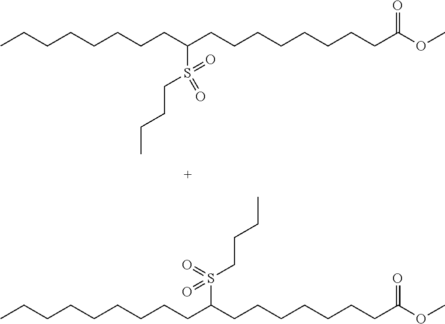

Chemical formula: C₂₃H₄₆O₄S

Molecular weight: 418.67 g/mol

Methyl oleate (130 g, 0.44 mol), butanethiol (470 ml, 4.4 mol) and AIBN (7.2 g, 0.044 mol) were introduced into a 1 l round-bottom flask and heated to 70° C. for 10 h. The reaction mixture, a yellowish liquid, was connected to a distillation apparatus, and then the butanethiol excess was distilled off. Then the by-product (dibutyl disulphide) was distilled off under reduced pressure until no dibutyl disulphide could be detected any longer by means of ¹H NMR. The remaining orange product was mixed in a 500 ml round-bottom flask with 30% by volume hydrogen peroxide (200 ml, 1.76 mol) and stirred vigorously at 70° C. for 15 h. The reaction mixture was transferred into a separating funnel and admixed with ethyl acetate (200 ml) and sodium chloride solution (200 ml). The aqueous layer was extracted and the organic layer was washed with 10% by weight aqueous Na₂SO₃ solution until the hydrogen peroxide content was below the detection limit of the Quantofix® peroxide test (below 1 mg/l). If necessary, for better phase separation, additional sodium chloride solution was added. The organic layer was dried over anhydrous Na₂SO₄ and filtered (if the solution remains turbid, it can be filtered through a 2 cm layer of silicon dioxide, in which case it is washed through with ethyl acetate), and then the solvent was drawn off on a rotary evaporator under reduced pressure. Product 7 was obtained in the form of a yellowish liquid.

FAB-MS [M+H]⁺ calculated: 419.67. found: 419.2.

Melting point: −3° C.

¹H NMR (300 MHz, CDCl₃, δ in ppm): δ=3.63 (s, OMe), 2.90-2.85 (m, —CH₂—S(O)₂—), 2.78-2.70 (m, —S(O)₂—CH<), 2.27 (t, J=7.50 Hz, —CH₂—CO—), 1.91-1.73 (m, —CH^{A}H^{B}—CHS(O)₂—CH^{A}H^{B}— and —CH₂—CH₂—S(O)₂—), 1.68-1.54 (m, —CH^{A}H^{B}—CHS(O)₂—CH^{A}H^{B}— and —CH₂—CH₂—CO—), 1.51-1.18 (m, —CH₂—), 0.93 (t, J=7.30 Hz, CH₃—CH₂—CH₂—CH₂—S(O)₂—), 0.84 (t, J=6.60 Hz, —CH₃).

### 2.3.10 Synthesis of a mixture of methyl 9-(hexylsulphonyl)octadecanoate/methyl 10-(hexylsulphonyl)octadecanoate (Product 8)

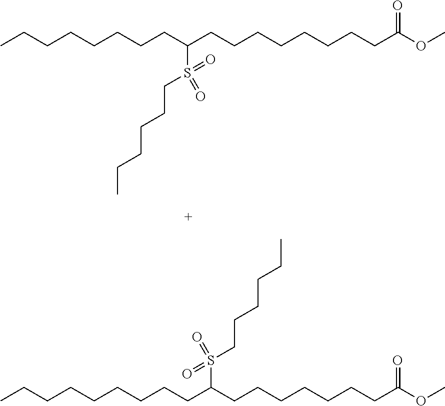

Chemical formula: C₂₅H₅₀O₄S

Molecular weight: 446.73 g/mol

Methyl oleate (125 g, 0.42 mol), hexanethiol (600 ml, 4.2 mol) and AIBN (6.9 g, 0.042 mol) were introduced into a 1 l round-bottom flask and heated to 70° C. for 10 h. The reaction mixture, a yellowish liquid, was connected to a distillation apparatus, and then the hexanethiol excess was distilled off under reduced pressure (20 mbar). Then the by-product (dihexyl disulphide) was distilled off under reduced pressure (about 0.01 mbar) until no dihexyl disulphide could be detected any longer by means of H NMR. The remaining orange product was mixed in a 1 l round-bottom flask with 30% by volume hydrogen peroxide (240 ml, 2.1 mol) and stirred vigorously at 70° C. for 24 h. The reaction mixture was transferred into a separating funnel and admixed with ethyl acetate (200 ml) and sodium chloride solution (200 ml). The aqueous layer was extracted and the organic layer was washed with 10% by weight aqueous Na₂SO₃ Solution until the hydrogen peroxide content was below the detection limit of the Quantofix® peroxide test (below 1 mg/l). If necessary, for better phase separation, additional sodium chloride solution was added. The organic layer was dried over anhydrous Na₂SO₄ and filtered (if the solution remains turbid, it can be filtered through a 2 cm layer of silicon dioxide, in which case it is washed through with ethyl acetate), and then the solvent was drawn off on a rotary evaporator under reduced pressure. Product 8 was obtained in the form of a yellowish liquid.

FAB-MS [M+H]⁺ calculated: 447.73. found: 447.3.

Melting points: −17° C., −13° C.

¹H NMR (300 MHz, CDCl₃, δ in ppm): δ=3.62 (s, OMe), 2.89-2.83 (m, —CH₂—S(O)₂—), 2.76-2.68 (m, —S(O)₂—CH<), 2.26 (t, J=7.50 Hz, —CH₂—CO—), 1.91-1.74 (m, —CH^{A}H^{B}—CHS(O)₂—CH^{A}H^{B}— and —CH₂—CH₂—S(O)₂—), 1.68-1.52 (m, —CH^{A}H^{B}—CHS(O)₂—CH^{A}H^{B}— and —CH₂—CH₂—CO—), 1.51-1.15 (m, —CH₂—), 0.88-0.81 (m, —CH₃).

Product 9: Synthesis of sulphone derivatives of fatty acid methyl esters based on rapeseed oil.

### Hydrolysis to the Free Fatty Acids:

150 g of rapeseed oil (˜0.17 mol, vita Rapsöl from Brändle), 50 g of NaOH (1.25 mol), 200 ml of THF and 120 ml of water (6.70 mol) were stirred in a 1 l round-bottom flask and heated under reflux for 3 hours. The reaction mixture was then cooled down with an ice bath and acidified with 15% hydrochloric acid to a pH of 1. The product was then neutralized by washing three times (1. diethyl ether, 2. 10 percent by weight NaHCO₃ solution, 3. 10 percent by weight NaCl solution). The organic phase was then dried over anhydrous Na₂SO₄ and filtered. The solvent was removed under reduced pressure with a rotary evaporator. The product is a pale orange liquid.

After derivatization of the free fatty acids to the corresponding methyl esters, a GC-MS analysis was conducted. The following composition was found: 82.3% oleic acid, 12.4% linoleic acid, 3.9% palmitic acid and 1.3% stearic acid.

### Thiol Ene Addition:

The thiol ene addition was conducted with ethanethiol under identical conditions to those for the technical grade methyl oleate (section 2.3.3). 11 equivalents of ethanethiol and 10 mol % of AIBN were used. A full conversion was obtained after 8 hours of reaction time. The excess of ethanethiol and any by-products formed was removed by means of fractional distillation. In this way, the thioether obtained from rapeseed oil was obtained.

### Oxidation to the Sulphones:

200 g of the thioether (0.58 mol) and one equivalent of 30% by volume hydrogen peroxide (65 ml, 0.58 mol) were stirred in a 500 ml round-bottom flask with reflux condenser for 30 min. After the exothermic reaction had abated, three further equivalents of hydrogen peroxide were added (195 ml, 1.74 mol). The reaction solution was heated under reflux for 8 h.

After phase separation, the aqueous phase was discarded. Subsequently, a further 4 equivalents of hydrogen peroxide (260 ml, 2.32 mol) were added and the reaction mixture was heated under reflux for a further 8 h. The aqueous phase was discarded. The organic phase was taken up in 300 ml of ethyl acetate and washed with 10% Na₂SO₃ until no peroxide was detectable any longer with a quick tester (Quantofix® peroxide test (below 1 mg/l)). The organic phase was dried over anhydrous Na₂SO₄ and filtered. The solvent was removed under reduced pressure with a rotary evaporator.

### Esterification to the Methyl Ester

150 g of the sulphone (˜0.40 mol) were mixed in a 1 l flask with 640 ml of methanol (15.90 mol), 4.2 g of trimethyl orthoformate (˜40 mmol) and 0.78 g of sulphuric acid (8 mmol). The reaction mixture was heated under reflux for 5 h. The excess methanol was removed under reduced pressure with a rotary evaporator. The yellowish residue was taken up in diethyl ether and neutralized by washing with a 10% NaHCO₃ solution. After a further wash step with NaCl solution, the organic phase was dried over anhydrous NaSO₄. Subsequently, the solvent was removed under reduced pressure by means of a rotary evaporator.

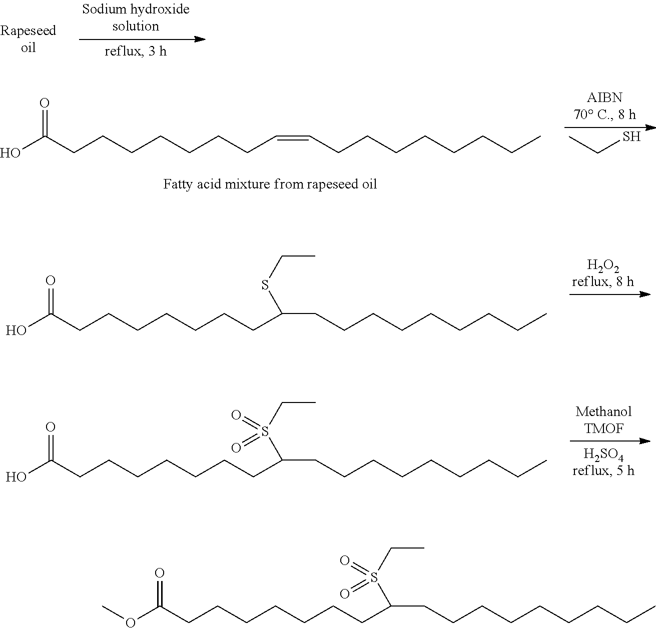

### Comparative Experiments for Plastisol Use:

### 1. Volatility

The volatility of plasticizers is a central property for many polymer applications. High volatilities lead to environmental exposure, and to worsened mechanical properties through reduced plasticizer contents in the polymer. Volatile plasticizers are therefore frequently added only in small proportions to other plasticizer systems or are not used at all. Volatility is of particular significance, for example, in interior applications (carpets, automobiles), or in cables or food packaging on the basis of guidelines and standards. The volatility of the pure plasticizers was determined with the aid of the Mettler Toledo HB 43-S halogen dryer. Before the measurement, a clean empty aluminium pan was positioned in the weighing pan. Thereafter, the aluminium pan was tared with a mat, and about five grams of plasticizer were pipetted onto the mat and weighed accurately.

The closure of the heating module started the measurement, and the sample was heated at maximum heating rate (preset) from room temperature to 200° C., and the corresponding loss of mass through vaporization was determined automatically by weighing every 30 seconds. After min, the measurement was ended automatically by the instrument.

A double determination was conducted on each sample.

The results are shown in Table 1. The plasticizer number (P No.) correlates here with the formulation number from Table 2.

TABLE 1

| | P No. | | | | | | | |
|---|---|---|---|---|---|---|---|---|
| | 1 | 2 | 3 | 4* | 5 | 6 | 7* | 8* |
| Loss of mass [%] | 5.4 | 14.6 | 45.2 | 4.6 | 16.2 | 50.8 | 5.8 | 3.8 |

*inventive ester

The thiols and sulphoxides (2, 3, 5, 6) show a distinctly increased loss of mass (>14% by weight) compared to DINP (1) and the sulphones (4, 7, 8). The inventive plasticizers (4, 7, 8), in contrast, actually show a slightly reduced or comparable volatility compared to DINP. The high volatility of the sulphoxides is attributable to a low thermal stability. This is a serious disadvantage for PVC applications, since temperatures up to 200° C. are typical for flexible PVC processing.

### 2. Production of the Plastisol

A PVC plastisol as used, for example, for production of topcoat films for floor coverings was produced. The figures in the plastisol formulations are each in parts by weight. The PVC used was Vestolit B 7021-Ultra. The comparative substance used was diisononyl phthalate (DINP, VESTINOL 9 from Evonik Industries). The formulations of the polymer compositions are listed in Table 2.

TABLE 2

| Formulation: | 1 | 2 | 3 | 4* | 5 | 6 | 7* | 8* |
|---|---|---|---|---|---|---|---|---|
| B 7021 -- Ultra | 100 | 100 | 100 | 100 | 100 | 100 | 100 | 100 |
| DINP | 50 | | | | | | | |
| Mixture of methyl 9- | | 50 | | | | | | |
| (ethylthio)octadecanoate/methyl 10- | | | | | | | | |
| (ethylthio)octadecanoate (Product 1) | | | | | | | | |
| Mixture of methyl 9- | | | 50 | | | | | |
| (ethylsulphinyl)octadecanoate/methyl 10- | | | | | | | | |
| (ethylsulphinyl)octadecanoate (Product 2) | | | | | | | | |
| Mixture of methyl 9- | | | | 50 | | | | |
| (ethylsulphonyl)octadecanoate/methyl 10- | | | | | | | | |
| (ethylsulphonyl)octadecanoate (Product 3) | | | | | | | | |
| Mixture of methyl 9,12- | | | | | 50 | | | |
| bis(ethylthio)octadecanoate/methyl 9,13- | | | | | | | | |
| bis(ethylthio)octadecanoate/methyl 10,12- | | | | | | | | |
| bis(ethylthio)octadecanoate/methyl 10,13- | | | | | | | | |
| bis(ethylthio)octadecanoate (Product 4) | | | | | | | | |
| Mixture of methyl 9,12- | | | | | | 50 | | |
| bis(ethylsulphinyl)octadecanoate/methyl 9,13- | | | | | | | | |
| bis(ethylsulphinyl)octadecanoate/methyl 10,12- | | | | | | | | |
| bis(ethylsulphinyl)octadecanoate/methyl 10,13- | | | | | | | | |
| bis(ethylsulphinyl)octadecanoate (Product 5) | | | | | | | | |
| Mixture of methyl 9,12- | | | | | | | 50 | |
| bis(ethylsulphonyl)octadecanoate/methyl 9,13- | | | | | | | | |
| bis(ethylsulphonyl)octadecanoate/methyl | | | | | | | | |
| 10,12-bis(ethylsulphonyl)octadecanoate/methyl | | | | | | | | |
| 10,13-bis(ethylsulphonyl)octadecanoate | | | | | | | | |
| (Product 6) | | | | | | | | |
| Mixture of methyl 9- | | | | | | | | 50 |
| (butylsulphonyl)octadecanoate/methyl 10- | | | | | | | | |
| (butylsulphonyl)octadecanoate (Product 7) | | | | | | | | |
| Drapex 39 | 3 | 3 | 3 | 3 | 3 | 3 | 3 | 3 |
| Mark CZ 149 | 2 | 2 | 2 | 2 | 2 | 2 | 2 | 2 |

*Polymer composition comprising an inventive ester

In the formulations, the product numbers correspond to those from the synthesis methods in section 2.3.1.

As well as the 50 parts by weight of plasticizer, each formulation also contains 3 parts by weight of an epoxidized soybean oil as a co-stabilizer (Drapex 39, from Galata), and 2 parts by weight of a Ca/Zn-based thermal stabilizer (Mark CZ 149, from Galata).

The plasticizers were equilibrated to 25° C. before the addition. First the liquid and then the pulverulent constituents were weighed into a PE beaker. The mixture was stirred in manually with an ointment spatula such that no unwetted powder was present any longer. The mixing beaker was then clamped into the clamp device of a dissolver stirrer. Before the immersion of the stirrer into the mixture, the speed was set at 1800 revolutions per minute. After the stirrer had been switched on, the mixture was stirred until the temperature on the digital display of the thermal sensor reached 30.0° C. This ensured that the homogenization of the plastisol was achieved with a defined energy input. Thereafter, the plastisol was equilibrated immediately at 25.0° C. In the case of the thioethers and sulphoxides, significant odour nuisance already occurred in the paste production. Because of the odour and the low thermal stability, the sulphoxides of fatty acid derivatives are not suitable as plasticizers. Consequently, they are no longer included in the further studies.

### 3. Measurement of the Plastisol Viscosities

The measurement of the viscosity of the PVC plastisols was conducted with a Physica MCR 101 (from Anton-Paar), using rotation mode and the “CC27” measurement system.

The plastisol was first homogenized once again in the mixing vessel by stirring with a spatula, then introduced into the measurement system and analysed isothermally at 25° C. During the measurement, the following actions were executed:

1. Preliminary shear at 100 s⁻¹ for a period of 60 s, in which no measurements were recorded (to level out any thixotropic effects which occur).
2. A downward shear rate ramp beginning at 200 s⁻¹ and ending at 0.1 s⁻¹, divided into a logarithmic series having 30 steps each having a measurement point duration of 5 seconds.

The measurements were generally (unless stated otherwise) conducted after storage/maturing of the plastisols for 24 h. Between the measurements, the plastisols were stored at 25° C.

In Table 3 below, the viscosities are given for each of the PVC pastes at a shear rate of 100 s⁻¹.

The paste number correlates here to the formulation number from Table 2.

TABLE 3

| | Paste No. | | | | | |
|---|---|---|---|---|---|---|
| | 1 | 2 | 4* | 5 | 7* | 8* |
| Paste viscosity after 24 h | 6.0 | 0.6 | 11.2 | 1.2 | 101.3 | 14.1 |
| (100 s⁻¹) in Pas | | | | | | |

*Pastes comprising an inventive ester

Compared to the DINP (1) and the thioethers (2, 5), pastes of the inventive fatty acid esters (4, 8) have a slightly elevated paste viscosity. The linoleic acid-based fatty acid ester (7), in contrast, has a distinctly increased paste viscosity at more than 100 Pas.

### 4. Gelling Characteristics

The study of the gelling characteristics of the pastes was undertaken in the Physica MCR 101 in oscillation mode with a plate-plate measuring system (PP25) which was operated under shear stress control. An additional temperature control hood was attached to the instrument in order to achieve a homogeneous heat distribution and a uniform sample temperature.

The following parameters were set:

Mode: Temperature gradient
Start temperature: 25° C.
End temperature: 180° C.
Heating/cooling rate: 5° C./min
Oscillation frequency: 4-0.1 Hz ramp (logarithmic)
Angular frequency omega: 10 1/s
Number of measurement points: 63
Measurement point duration: 0.5 min
Automatic gap readjustment F: 0 N
Constant measurement point duration
Gap width 0.5 mm

### Measurement Procedure:

A drop of the plastic to be analysed was applied, free of air bubbles, with a spatula to the lower measurement system plate. In doing this, it was ensured that, after the closure of the measurement system, some paste could exude uniformly out of the measurement system (not more than about 6 mm overall). Subsequently, the temperature control hood was positioned over the sample and the measurement was started. The “complex viscosity” of the paste was determined as a function of temperature. Since a particular temperature is attained within a time span (fixed by the heating rate of 5° C./min), not only the gelation temperature but also a statement about the gelling rate of the system analysed is obtained. Onset of the gelling process was recognizable by a sudden sharp rise in the complex viscosity. The earlier the onset of this viscosity rise, the better the gelatability of the system.

The cross-over temperature is determined from the measurement curves obtained. This method calculates the point of intersection of the two selected y variables. It is used to find the end of the linear-viscoelastic range in an amplitude sweep (y: G′, G″; x: gamma), to find the crossing frequency in a frequency sweep (y: G′, G″; x: frequency) or to determine the gelling time or curing temperature (y: G′, G″; x: time or temperature). The cross-over temperature documented here corresponds to the temperature of the first point of intersection of G′ and G″.

The results are shown in Table 4. The paste number correlates here with the formulation number from Table 2.

TABLE 4

| | Paste No. | | | | | |
|---|---|---|---|---|---|---|
| | 1 | 2 | 4* | 5 | 7* | 8* |
| Cross-over temperature ° C. | 78 | 87 | 68 | 87 | 75 | 69 |

*Pastes comprising an inventive ester

Compared to the paste comprising DINP (1), pastes 2 and 5 comprising a thioether compound have a distinctly increased crossover temperature. This is equivalent to slowed gelling. The polymer compositions comprising an inventive plasticizer (paste Nos. 4, 7 and 8), in contrast, have faster gelling than DINP.

Because of the odour nuisance and the slowed gelling, the thioethers of fatty acid derivatives (samples 2 and 5) are only of very limited suitability as plasticizers. They are consequently no longer included in the further studies.

For further studies on flexible PVC specimens, fully gelated 1 mm polymer films were produced from the corresponding plastisols (gelling conditions in the Mathis oven: 200° C./2 min.).

### 5. Plasticizing Action

The Shore hardness is a measurement of the softness of a specimen. The further a standardized needle can penetrate into the specimen in a particular measurement duration, the lower is the measurement. The plasticizer having the highest efficiency gives the lowest value for the Shore hardness for the same amount of plasticizer. Since formulations/recipes are in practice frequently adjusted or optimized for a particular Shore hardness, it is accordingly possible in the case of very efficient plasticizers to dispense with a particular proportion in the formulation, which means a reduction in costs for the processor.

To determine the Shore hardnesses, the pastes produced as described above were poured into round brass casting moulds having a diameter of 42 mm (weight: 20.0 g). Then the pastes were gelled in the moulds in the forced-air drying cabinet at 200° C. for 30 min, cooled and then removed and, prior to the measurement, stored in a climate-controlled cabinet (25° C.) for at least 24 hours. The thickness of the disks was about 12 mm.

The hardness measurements were conducted to DIN 53 505 with a Shore A measuring instrument from Zwick-Roell; the measurement was read off after 3 seconds in each case. Measurements were conducted at three different sites on each specimen, and a mean was formed.

The results are shown in Table 5. The specimen number correlates here with the formulation number from Table 2.

TABLE 5

| Specimen No. | 1 | 4* | 8* |
|---|---|---|---|
| Shore A | 80 | 72 | 77 |

Compared to the industry standard, DINP (specimen 1), specimens 4 and 8 have Shore hardnesses lower by 3.7% and 10% respectively. With the inventive plasticizers, it is possible to produce PVC mixtures having better efficiency than in the case of use of the corresponding DINP. It is thus possible to save plasticizer, which leads to lower formulation costs.

1: An ester compound of formula I: 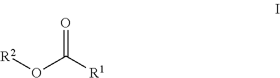
wherein:
R¹ is a carbon chain having 7 to 25 carbon atoms, and the carbon chain R¹ has at least one sulfone group, and
R² is a linear or branched radical having 1 to 12 carbon atoms,
and wherein the sulfur atom in the sulfone group is bonded directly to a carbon atom in the carbon chain R¹, and the carbon atom to which the sulfone group is directly bonded is not the carbon atom furthest removed from the C═O group, and where the sulfone group has the following structure: 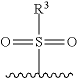
where R³ is an aliphatic radical having 1 to 8 carbon atoms.

2: The ester compound of claim 1, wherein R² is a linear or branched radical having 1 to 9 carbon atoms.

3: The ester compound of claim 1, wherein R¹ is a carbon chain having 11 to 21 carbon atoms.

4: The ester compound of claim 1, wherein R¹ is a carbon chain having 17 carbon atoms.

5: The ester compound of claim 1, wherein R³ is an aliphatic radical having 1 to 4 carbon atoms.

6: The ester compound of claim 1, wherein the carbon chain R¹ has exactly one sulfone group.

7: The ester compound of claim 1, having the following structure: 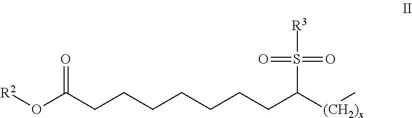
wherein x is a number from 6 to 12.

8: The ester compound of claim 1, having the following structure: 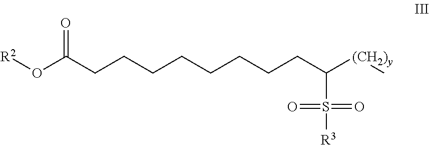
wherein y is a number from 5 to 11.

9: An ester mixture, comprising the ester compound of claim 7, and comprising an ester compound having the following structure: 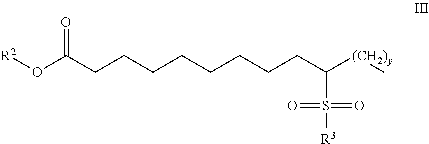
wherein y is a number from 5 to 11.

10: The ester mixture of claim 9, wherein acid components of the ester mixture are obtained from a vegetable oil.

11: A composition comprising the ester compound of claim 1 as a plasticizer, and a polymer comprising at least one member selected from the group consisting of polyvinyl chloride, polyvinylidene chloride, polylactic acid, a polyurethane, polyvinyl butyral, and a polyalkyl methacrylate.

12: A composition comprising the ester compound of claim 1 as a plasticizer, and polyvinyl chloride.

13: A process for preparing the ester compound of claim 1, comprising:
a) initially charging an unsaturated fatty acid or fatty acid mixture or a fatty acid ester or fatty acid ester mixture,
b) adding a thiol onto the double bond, so as to form a thioether,
c) oxidizing the thioether to the sulfone, and
d) esterifying the fatty acid or fatty acid mixture or transesterifying the fatty acid ester or fatty acid ester mixture.

14: The process of claim 13, wherein the adding b) is effected with addition of a free-radical initiator.

15: The process of claim 13, wherein the oxidizing c) is effected with addition of H₂O₂.
